(19) United States
(12) Patent Application Publication
(10) Pub. No.: US 2021/0332319 A1
(43) Pub. Date: Oct. 28, 2021

(54) CLEANING SYSTEMS FOR BIOREACTORS

(71) Applicant: Hypergiant Industries, Inc., Austin, TX (US)

(72) Inventors: Benjamin Edward Lamm, Dallas, TX (US); Andrew Thomas Busey, Austin, TX (US); Daniel David Haab, Austin, TX (US); Davis Michael Saltzgiver, Austin, TX (US); Willem Vonk, Austin, TX (US)

(21) Appl. No.: 17/237,238

(22) Filed: Apr. 22, 2021

Related U.S. Application Data
(60) Provisional application No. 63/014,507, filed on Apr. 23, 2020.

(51) Int. Cl. C12M 1/00; B08B 9/055; F16L 45/00

(52) U.S. Cl. CPC C12M 39/00; B08B 9/055; B08B 2209/045; B08B 2209/055; F16L 45/00

(57) ABSTRACT

Systems and methods for cleaning a photobioreactor apparatus are described. Cleaning devices are described that include flexible members coupled to a propulsion member. The cleaning devices may be moved through tubes of the photobioreactor apparatus using the propulsion member. As the cleaning devices move, the flexible members may make contact with walls of the tubes, thereby cleaning the walls of the tubes. Cleaning devices are also contemplated that include particles with some rigidity that intermix with the fluid inside the tubes and clean the walls of the tubes as the particles contact the walls.

### PRIORITY CLAIM

This patent application claims priority to U.S. Provisional Patent Application No. 63/014,507, filed Apr. 23, 2020, which is incorporated by reference as if fully set forth herein.

### BACKGROUND

### 1. Field of the Invention

The present disclosure relates generally to devices for producing biological organisms. More particularly, embodiments disclosed herein relate to systems and methods for cleaning and maintenance of devices, such as photobioreactors, that support the production of microorganisms such as algae.

### 2. Description of Related Art

Photobioreactors are reactors that utilize a light source to support the growth of phototrophic microorganisms in a controlled, artificial environment. Photobioreactors may be used to support photosynthetic growth of various different organisms using carbon dioxide and light. Examples of organisms that have been grown using photobioreactors include algae (e.g., macroalgae and/or microalgae), plants, mosses, cyanobacteria, and purple bacteria.

### BRIEF DESCRIPTION OF THE DRAWINGS

Features and advantages of the methods and apparatus of the embodiments described in this disclosure will be more fully appreciated by reference to the following detailed description of presently preferred but nonetheless illustrative embodiments in accordance with the embodiments described in this disclosure when taken in conjunction with the accompanying drawings in which:

FIG. 1 depicts an isometric view of an embodiment of a bioreactor.

FIG. 2 depicts an exploded isometric view of an embodiment of a bioreactor showing components of a top manifold and a bottom manifold.

FIG. 3 depicts an enlarged, exploded isometric view of an embodiment of the components in a top manifold.

FIG. 4 depicts an enlarged, exploded isometric view of an embodiment of the components in a bottom manifold.

FIG. 5 depicts an enlarged, exploded isometric view of an embodiment of the components in a bottom manifold that is rotated 180° from the view depicted in FIG. 4.

FIG. 6 depicts an exploded perspective view of an embodiment of a bioreactor showing fluid flow.

FIG. 7 depicts a representation of a cleaning device, according to some embodiments.

FIG. 8 depicts a representation of cleaning device, according to some embodiments.

FIGS. 9A and 9B depict a representation of an example of controlling movement of a cleaning device in a tube using gravity and flow, according to some embodiments.

FIG. 10 depicts a representation of an example of controlling movement of a cleaning device in a tube with fluid flow, according to some embodiments.

FIG. 11 depicts a representation of an example of controlling movement of a cleaning device in a tube with a magnetic device, according to some embodiments.

FIG. 12 depicts a representation of a cleaning device system, according to some embodiments.

### DETAILED DESCRIPTION OF EMBODIMENTS

Photobioreactors are used as controlled, artificial environments for the growth of microorganisms. As used herein, a “photobioreactor” refers to reactor that utilizes a light source to promote growth of phototrophic microorganisms. In many instances, photobioreactors support photosynthetic growth of microorganisms in a fluid using carbon dioxide and light. Microorganisms that may be grown in photobioreactors include, but are not limited to, algae (e.g., macroalgae and/or microalgae), plants, mosses, cyanobacteria, and purple bacteria.

Photobioreactors can include either open systems or closed systems. Open systems are typically used for producing phototrophic organisms on an industrial scale. Open systems, however, require large areas and large water sources and may have limited productivity rates and high losses due to water evaporation. Closed systems may provide more controllable growth. Closed systems, however, may be more expensive or more difficult to operate for producing phototrophic organisms on an industrial scale.

As described herein, the use of tubes in a bioreactor may provide efficient growth of biological organisms. While tubes provide efficient growth of biological organisms, the efficient growth may necessitate that the tubes have to be cleaned or maintained on a more regular basis to remove biological organisms growing on the inner walls of the tubes. If the inner walls of the tubes are not cleaned frequently enough, growth of the biological organisms on the inner walls may restrict or block light from reaching biological organisms inside the tubes. Thus, there is a need for devices and methods to efficiently clean biological organisms off the inside walls of tubes.

A current method for cleaning biological organisms off the walls of tubes includes flushing fluid from the tubes, opening the tubes, and scrubbing the tubes to remove biological organism growth from the walls. This cleaning method, however, may be time consuming and also generates downtime for a bioreactor (e.g., time where the bioreactor is not used to grow biological organisms). This downtime reduces the productivity of growing biological organisms using the bioreactor.

A productive method for cleaning tubes may be the use of a peg. As used herein, a “peg” refers to an object that can move through tubes and clean the walls of the tubes as the peg moves along the tubes. The present inventors have recognized that pegs may be designed with features that provide efficient cleaning of tubes and reduce downtime for a bioreactor. For example, the inventors have recognized that pegs may be designed with features that allow a bioreactor to be continuously operated to grow biological organisms while the pegs are used to clean the tubes.

The present disclosure contemplates devices, and related methods, that are configured to clean conduits (e.g., tubes) associated with growing biological organisms in a bioreactor. Embodiments described herein include devices that make contact with walls of the conduits, move along the walls of the conduits without getting stuck or stopping for an undesirable time, losing orientation, or impeding flow of fluid through the conduits in a negative way. Some embodiments also include devices that are controllable either manually or automatically. For example, movement of the devices can be controlled by controlling the flow of fluid in the conduits.

One embodiment described herein has two broad components: 1) a flexible member, and 2) at least one propulsion member coupled to the flexible member. In certain embodiments, the flexible member has a circular edge. The flexible member may flex outwards such that the circular edge makes contact with an inner wall of a conduit for cleaning the conduit. In various embodiments, the flexible member includes at least one opening to allow fluid flow through the flexible member. In various embodiments, the at least one propulsion member includes at least one surface that provides resistance against the fluid flow in order to propel the device in a direction of the fluid flow. In some embodiments, the propulsion member has a buoyancy that allows the device to float on a surface of a fluid. As such, the propulsion member can be moved through a conduit by changing a fluid level in the conduit. In some embodiments, the propulsion member may be moved by changing a direction of flow in the conduit. In various embodiments, a magnet is coupled to the propulsion member and an external magnet is used to move the propulsion member in the conduit.

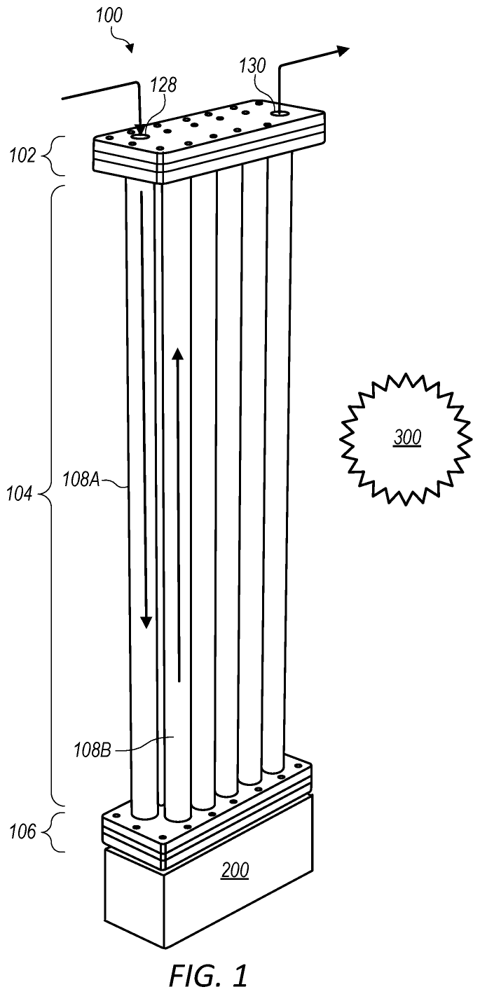

FIG. 1 depicts a perspective view of an embodiment of bioreactor 100. In certain embodiments, bioreactor 100 is a modular bioreactor. A modular bioreactor 100 may, for example, be coupled to one or more additional bioreactors to form up a larger bioreactor. In such embodiments, bioreactor 100 includes connections that allow multiple bioreactors to be coupled together. In some contemplated embodiments, multiple bioreactors 100 are coupled together in series to form a single, larger bioreactor with single output of organisms. In other contemplated embodiments, multiple bioreactors 100 are coupled together in parallel to provide multiple parallel outputs of organisms.

In the illustrated embodiment, bioreactor 100 includes top manifold 102, tube section 104, and bottom manifold 106. Tube section 104 may include a plurality of tubes 108 coupled between top manifold 102 and bottom manifold 106. Tubes 108 may be made of glass, plastic, or any other material that is substantially transparent to a desired spectrum of light (e.g., a visible spectrum light). Top manifold 102 and bottom manifold 106 may direct (e.g., route) the flow of fluid through tubes 108 (e.g., direct fluid flow from one tube to the next).

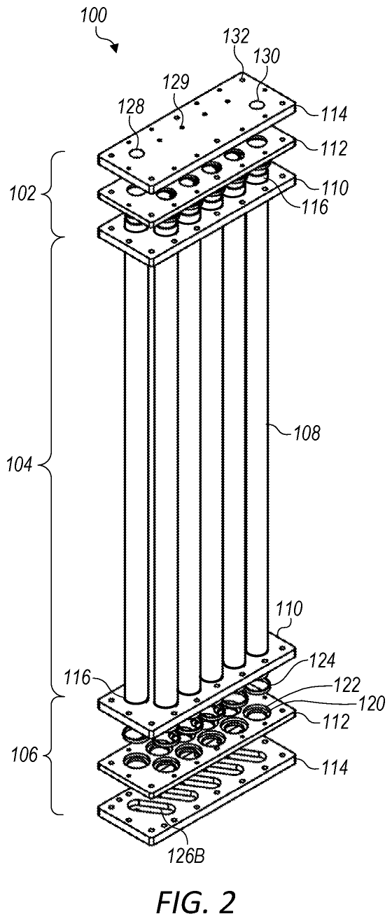

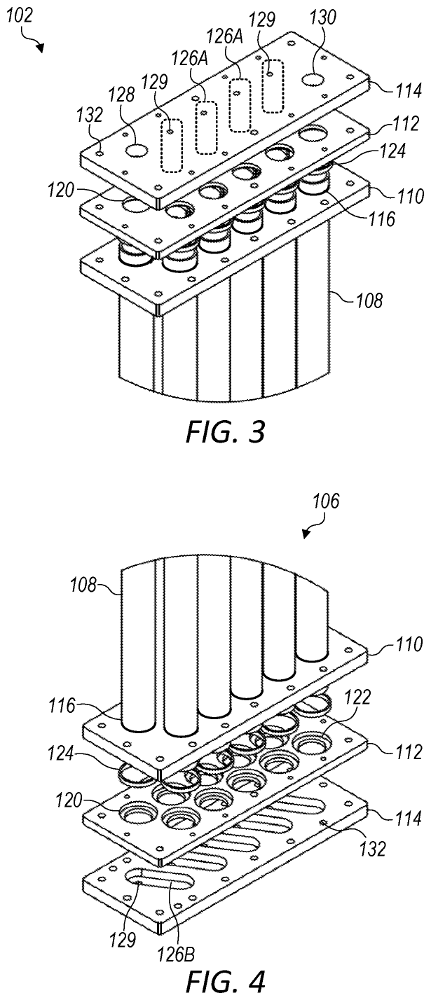

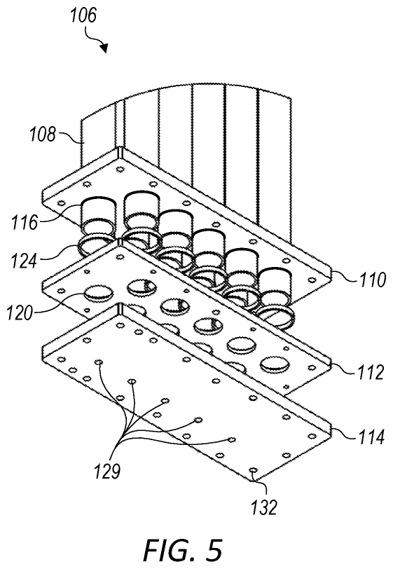

FIG. 2 depicts an exploded isometric view of an embodiment of bioreactor 100 showing components of top manifold 102 and bottom manifold 106. FIG. 3 depicts an enlarged, exploded isometric view of an embodiment of the components in top manifold 102. FIG. 4 depicts an enlarged, exploded isometric view of an embodiment of the components in bottom manifold 106. FIG. 5 depicts an enlarged, exploded isometric view of an embodiment of the components in bottom manifold 106 that is rotated 180° from the view depicted in FIG. 4. In certain embodiments, the components of top manifold 102 and bottom manifold 106 include capture plates 110, guide plates 112, and interface plates 114.

As shown in FIGS. 2-5, ends of tubes 108 may be inserted through capture plates 110 in both top manifold 102 and bottom manifold 106. Tubes 108 can be inserted through holes 116 in capture plates 110. Holes 116 may be sized such that tubes 108 have a substantially secure fit (e.g., tight fit) within the holes.

After tubes 108 pass through capture plates 110, the tubes may be inserted through holes 120 in guide plates 112. In certain embodiments, guide plates 112 include recesses 122 at holes 120 (as shown in FIGS. 2 and 4). Recesses 122 may be shaped to seat o-rings 124 in guide plates 112. O-rings 124, when seated in recesses 122, may form a seal between the outside surface of tubes 108 and the surfaces of guide plates 112 as the tubes pass through the guide plates. The seal formed may inhibit fluid moving between adjacent tubes 108 in interface plates 114 (as described below) from leaking outside the manifolds. Friction between tubes 108 and o-rings 124 along with friction between the o-rings and the plates may hold the tubes within the manifolds. Using only friction to hold tubes 108 in place may allow the tubes to be removed for maintenance and/or replacement, as described herein. In some embodiments, holes 116 and/or holes 120 may be sized to allow for variations in the diameter of tubes 108. Tubes 108 can have variations in diameter due to variances in manufacturing of the tubes. Thus, holes 116 and/or holes 120 may be sized to accommodate such manufacturing variances.

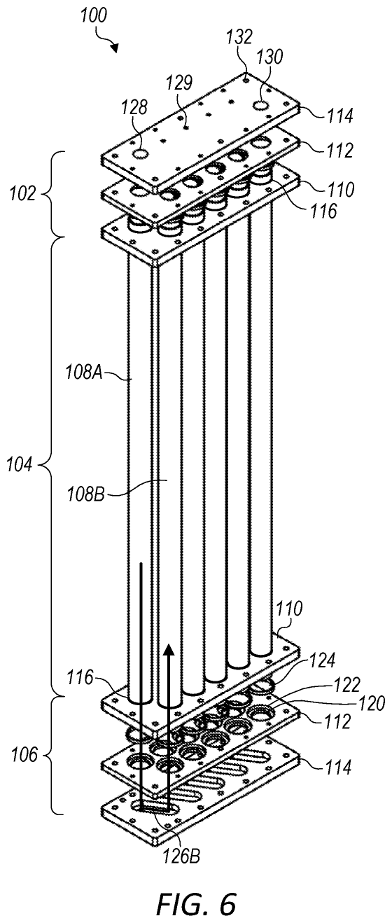

Ends of tubes 108 may be positioned in recesses 126 in interface plates 114. For example, at least a portion of tubes 108 are placed within recesses 126. Recesses 126 may be grooved recesses or other indentions in interface plates 114 that act as passages to allow fluid communication between two tubes 108 when the ends of the tubes are positioned in the recesses. As such, fluid may flow out an end of a first tube and into the end of a second tube when the ends of the tubes are positioned in recesses 126 (e.g., flow is directed from one tube to the next tube by the recesses). FIG. 6 depicts an exploded perspective view of an embodiment of bioreactor 100 showing fluid (represented by the arrow) exiting tube 108A, moving through recess 126, and going up tube 108B.

In certain embodiments, recesses 126A (shown by dashed lines in FIG. 3) in top manifold 102 and recesses 126B (shown in FIGS. 2 and 4) in bottom manifold 106 are oriented in opposing directions such that fluid flow is directed through tubes 108 in series (e.g., sequentially from one tube to the next) between inlet 128 and outlet 130. For example, recesses 126A and recesses 126B may be oriented perpendicular or close to perpendicular with respect to each other. Orienting recesses 126A and recesses 126B in this manner may direct fluid in a single direction through each of tubes 108 between inlet 128 and outlet 130. Thus, as shown by the arrows in FIG. 1, fluid may enter bioreactor 100 at inlet 128, go down first tube 108A, then up second tube 108B, and continue this pattern to outlet 130. Directing fluid through each of tubes 108 may route the fluid in a linear way and make one continuous flow path for fluid through the tubes. Providing the one continuous flow path through tubes 108 in bioreactor 100 may maximize the surface area in contact with the fluid in the bioreactor for the growth of biological organisms in the bioreactor.

While the embodiment of bioreactor 100 shown in FIGS. 1-5 depicts tubes 108 arranged in a series configuration (flow from one tube to the next), other embodiments may be contemplated where tubes 108 are arranged in a parallel configuration. For example, recesses 126 in top manifold 102 and/or bottom manifold 106 may be positioned such that tubes 108 are coupled to a tank, harvester, or other external apparatus in parallel. Connecting tubes 108 in parallel may provide direct feedback between the external apparatus and the tubes.

In various embodiments, routing fluid through inlet 128, tubes 108, and outlet 130, as shown in FIG. 1, may provide modularity for the design of bioreactor 100 and allow the bioreactor to be coupled to one or more additional bioreactors as part of a group of bioreactors. In certain embodiments, both inlet 128 and outlet 130 are positioned in a single manifold (e.g., top manifold 102). For example, with an even number of tubes 108, inlet 128 and outlet 130 may be positioned in the same manifold. Other embodiments with odd numbers of tubes may also be contemplated. In embodiments with odd numbers of tubes 108, inlet 128 and outlet 130 may be positioned in different manifolds (e.g., the inlet is in top manifold 102 and the outlet is in bottom manifold 106).

In certain embodiments, capture plates 110, guide plates 112, and interface plates 114 are made of high-density materials that inhibit leaking. For example, in some embodiments, capture plates 110, guide plates 112, and interface plates 114 are made of polycarbonate and/or HDPE (high-density polyethylene). In some embodiments, capture plates 110, guide plates 112, and interface plates 114 are made of metals such as, but not limited to, aluminum. Using metal materials may provide more rigidity and reduce chances for breakage and/or leakage from the manifolds.

In certain embodiments, capture plates 110, guide plates 112, and interface plates 114 may be held together using fasteners 132. Fasteners 132 may be, for example, screws, bolts, or other fastener devices. Fasteners 132 may be distributed around the edges of the plates to distribute the clamping forces around the plates. In some embodiments, capture plates 110, guide plates 112, and interface plates 114 may be held together using a clamp-type device. The clamp-type device may include one or more latches to secure the plates together. The latches may allow the plates to be repeatably secured and unsecured for cleaning and/or other operations (e.g., removal of broken tubes from the manifolds). In some embodiments, the plates are hinged (e.g., the plates may be hinged together on one side of the plates). Hinging the plates may allow the plates to be opened and closed without separation of the plates.

In certain embodiments, one or more gaskets (or another sealing material) are placed between the plates to provide a seal inhibiting fluid leakage from the manifolds. Gaskets may be used, for example, in combination with fasteners 132 and/or latches to provide sealing when the plates are secured together. In some embodiments, a sealant material (e.g., silicone) may be used to provide additional protection against leaks from the manifolds. For example, the sealant material may be placed around the outside of the manifold to prevent leakage of fluid therefrom.

In certain embodiments, interface plates 114 include drain holes 129. Drain holes 129 may be aligned and in fluid communication with recesses 126. Drain holes 129 may provide fluid access to tubes 108 through recesses 126. In some embodiments, bleed valves or drain valves may be coupled to drain holes 129. For example, bleed valves may be coupled to drain holes 129 in a manifold to bleed off gas (e.g., air) as tubes 108 are filled with fluid (e.g., water). Bleeding off gas may equalize pressure in tubes 108 as the tubes are filled and ensure proper filling of the tubes with fluid without trapping gas in the tubes. For example, in one embodiment, gas (air) may be pushed out of tubes 108 as fluid fills the tubes. In another embodiment, a pump or other suction device may be coupled to drain holes 129 to pull gas from the tubes until fluid fills up the tubes and begins to be drawn out through the drain holes. In some embodiments, drain valves may be coupled to drain holes 129 in a manifold to drain tubes 108 as needed. Providing individual drain holes 129 may provide for more controlled bleeding or draining of tubes 108.

In some embodiments, one or more components in top manifold 102 or bottom manifold 106 are integrated into a single component. For example, capture plates 110 and guide plates 112 may be integrated into a single component with o-rings 124 positioned inside the single component. In some embodiments, top manifold 102 or bottom manifold 106 may include access ports to access tubes 108. For example, a manifold may have screw caps at the positions of drain holes 129. The screw caps may be removable from the manifold to provide access to tubes 108. Seals may prevent leakage around the screw caps when in place on the manifold.

In some embodiments, one or more sensors are included in top manifold 102 or bottom manifold 106. Sensors may be used to assess operating properties of bioreactor 100. Operating properties assessed may include, but not be limited to, flow rate, temperature, pressure, pH, and photon detection. In some embodiments, sensors may be provided into tubes using the access ports described above. In some embodiments, sensors may be placed in a secondary reservoir attached to tubes 108 (e.g., a reservoir in utility system 200, described below).

The structures of top manifold 102 and bottom manifold 106 may also provide the ability for more simple cleaning and maintenance of bioreactor 100. For instance, a manifold may be opened (such as by opening the latches) to provide access to tubes 108 for cleaning or replacement of the tubes. If the manifold is permanently sealed (e.g., is sealed with silicone), the manifold may be removed to provide access for cleaning or replacement of tubes and may then be replaced with a new manifold.

Bioreactor 100 may be used to grow different types of biological organisms. In certain embodiments, bioreactor 100 is used to grow algae. The algae may include macroalgae and/or microalgae. Other biological organisms that may be grown using bioreactor 100 include, but are not limited to, plants, mosses, and bacteria (e.g., cyanobacteria or purple bacteria). Top manifold 102 and bottom manifold 106 provide structures that hold tubes 108 as close together as possible to produce a small footprint for bioreactor 100. In certain embodiments, tubes 108 have an average spacing between the tubes of at most about 0.5 inches. As used herein, “average spacing” refers to an average of the distances between outside walls of tubes 108 in bioreactor 100. The average spacing between tubes 108 may, however, vary. For example, larger spacings may be implemented to accommodate additional hardware or equipment in spaces between tubes 108 (such as hardware to allow the tubes to be more easily removable). In some embodiments, tubes 108 may have an average spacing between the tubes of between about 0.25 inches and about 0.5 inches, between about 0.25 inches and about 0.75 inches, or between about 0.1 inches and about 1.5 inches.

In some embodiments, tubes 108 have a length that varies between about 30 inches and about 70 inches. For example, tubes 108 may have a length of about 48 inches. Other lengths of tubes 108 may, however, also be contemplated depending on the requirements for growth of biological organisms in bioreactor 100. In some embodiments, tubes 108 have diameters that vary between 0.5 inches and 1.5 inches. In one embodiment, tubes 108 have diameters of 0.75 inches. Diameters of tubes 108 may also vary depending on the requirements for growth of biological organisms in bioreactor 100. For example, the lengths or diameters of tubes 108 may vary based on biological requirements that may be algae strain dependent.

The embodiment of bioreactor 100 illustrated in FIGS. 1-5 is a modular bioreactor that includes a high density of tubes 108 in a low-cost structure. Utilizing tubes 108 in bioreactor 100 provides an efficient way to grow biological organisms by increasing the surface area per volume of fluid that the organisms are growing in as compared to other typical bioreactors (e.g., open bioreactors). Increasing the surface area per volume of fluid using tubes 108 in a dense configuration may also provide a large amount of surface area for growth of biological organisms in a relatively small footprint. For example, in one embodiment, bioreactor 100 with ten tubes in a footprint of (6 inches×15 inches×48 inches) may have a combined light exposed surface area of about 2262 square inches and a combined volume of about 848 cubic inches, which gives about 4400 square inches of exposed algae per cubic foot. A rectangular volume bioreactor having the same footprint may only have an exposed surface area of about 2016 square inches with a volume of about 4320 cubic inches, which gives only about 1692 square inches of exposed algae per cubic foot. Thus, bioreactor 100 may provide a larger exposed algae area per cubic foot. In some embodiments, bioreactor 100 may have a surface area of exposed algae per cubic foot of at least about 2500 square inches per cubic foot, at least about 3000 square inches per cubic foot, at least about 4000 square inches per cubic foot, or at least about 5000 square inches per cubic foot. The surface area per cubic foot volume of the bioreactor may be varied by using longer or shorter tubes 108 or different diameter tubes to provide more surface area (longer tubes) or less surface area (shorter tubes) as desired.

Having multiple tubes 108 operating in series (as described above) in bioreactor 100 also may increase the efficiency of light energy (e.g., photons) reaching the growing biological organisms in the bioreactor. As such, bioreactor 100 provides an efficient biological organism growth apparatus in a small and modular size. The number of tubes 108 in bioreactor 100 may also be varied to produce different sizes of reactor modules as desired. Additionally, the modularity of bioreactor 100 may allow the bioreactor to be combined with additional bioreactor modules to form larger bioreactors.

In the illustrated embodiment of FIG. 1, utility system 200 is positioned near or coupled to a manifold in bioreactor 100. In certain embodiments, utility system 200 is attached to or positioned in a structure (e.g., a housing or cabinet) used to support the manifolds and tubes to provide a modular system for the bioreactor. Utility system 200 may include devices and/or apparatus that are used to facilitate growth of biological organisms in bioreactor 100. Examples of devices and/or apparatus included in utility system include, but are not limited to, fluid circulators (e.g., pumps), reservoirs (e.g., tanks), sensors, gas sources, nutrient (feedstock or raw material) feeders, and cleaning devices.

In certain embodiments, a reservoir in utility system 200 is in fluid communication with tubes 108 (e.g., through inlet 128 on a manifold (such as top manifold 102)). The reservoir may be a source of fluid and feedstock used for the growth of biological organisms in tubes 108. In some embodiments, a fluid circulator (e.g., a pump) is coupled to or placed in the reservoir. The fluid circulator may move fluid and feedstock to tubes 108 from the reservoir. In some embodiments, the reservoir may be an open-air reservoir that allows carbon dioxide to be pulled from the surrounding air.

In certain embodiments, utility system 200 includes a harvester. The harvester may, for example, be coupled to outlet 130 on a manifold (such as top manifold 102) and be in fluid communication with tubes 108 through the outlet. The harvester may be used to harvest biomass (e.g., a mass of biological organisms) grown from tubes 108.

In certain embodiments, utility system 200 is coupled to inlet 128 and outlet 130 on a manifold (e.g., top manifold 102). Tubes or valves may be used to couple utility system 200 to the manifold. In some embodiments, pumps or other fluid circulators in utility system provide pressure to create mixed flow in tubes 108 (e.g., mixing of biomass and fluid in the tubes). Mixing in tubes 108 may be used to inhibit settling of biomass in recesses 126 in the manifolds or to promote growth of biomass in the tubes.

In certain embodiments, bioreactor 100 includes light source 300. Light source 300 may be any light source capable of providing light in wavelengths suitable for growth of a desired biological organism in bioreactor 100. For example, light source 300 may provide light at visible wavelengths, UV wavelengths, near-UV wavelengths, or combinations thereof. Thus, light source may provide light at wavelengths between 100 nm and 700 nm or smaller ranges therein. In some embodiments, light source 300 is fluorescent lights or LED lights capable of visible, UV, or near-UV radiation. In some embodiments, light source 300 is attached or included as part of a structure (e.g., a housing or cabinet) used to support the manifolds and tubes of bioreactor 100. In some embodiments, light source 300 is external to the structure used to support the manifolds and tubes of bioreactor 100.

### Maintenance and Cleaning of Bioreactors

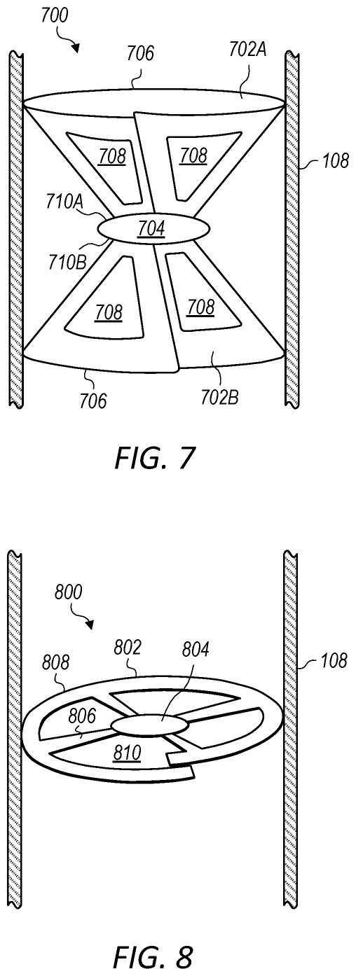

FIGS. 7-12 depict embodiments of devices configured to clean conduits (e.g., tubes 108) associated with growing biological organisms in bioreactor 100. FIG. 7 depicts a representation of cleaning device 700, according to some embodiments. Cleaning device 700 includes cleaning members 702 coupled to base member 704. In certain embodiments, cleaning members 702 include cleaning member 702A coupled to a first side (e.g., a front side) of base member 704 and cleaning member 702B coupled to a second side (e.g., a back side) of the base member. Cleaning members 702 may include, but not be limited to, cones, conical-shaped members, fins, or fin-shaped members. In some embodiments, cleaning device 700 has a “shuttlecock” design with cleaning members 702 and base member 704. For example, cleaning members 702 may have conical shapes that form the “feathers” of the shuttlecock design where an apex of the conical shape is coupled to base member 704 and the base member is a solid object that forms the “base” of the shuttlecock design. For instance, base member 104 may be a solid “ball”-shaped object. Ends of cleaning members 702 distal from the coupling between the cleaning members and base member 704 (e.g., the base of the conical shape) may have circular openings with edges 706 (e.g., circular edges).

In certain embodiments, cleaning members 702 are made of flexible materials that have some rigidity. For instance, cleaning members 702 may be soft plastic or another flexible, semi-rigid material. The flexibility of cleaning members 702 may allow the cleaning members to move freely through tubes 108 and flex or squish as the cleaning members encounter debris or objects in the tubes. In some embodiments, the flexibility of cleaning members 702 may additionally enhance cleaning of the walls as the cleaning members move through tubes 108. The component cleaning member 702 and its corresponding structural equivalents may be referred to as a “means for cleaning walls of a conduit”.

In certain embodiments, the rigidity of cleaning members 702 gives the cleaning members spring-like properties. For example, cleaning members 702 may spring or flex outwards towards the walls of tubes 108 because of the rigidity of the cleaning members. The spring or flex outwards may force contact between edges 706 of cleaning members 702 and the walls of tubes 108 (e.g., circular edges make contact with the walls along a circumference of the circular edges). Thus, as described herein, cleaning members 702 may expand outwards to have contact against the walls of tubes 108 due to their spring-like behavior while having flexibility (e.g., squishiness) that allows the cleaning members to freely move through the tubes.

In certain embodiments, cleaning members 702 include openings 708. Openings 708 may be openings in the material of cleaning members 702 that allow fluid to flow through cleaning members 702 and reduce drag on cleaning device 700. In some embodiments, openings 708 are sized, shaped, or include features (e.g., protrusions) to promote mixing of the fluid as the fluid flows through the openings. Promoting mixing may promote the growth of biological organisms in tubes 108. In some embodiments, openings 708 are sized or shaped to maintain an orientation of cleaning device 700 as the device moves through tubes 108. For example, openings 708 may maintain cleaning device 700 in an upright orientation, as shown in FIG. 7, based on drag and motion forces acting on the openings.

In some embodiments, a scrubbing material is positioned along edges 706 of cleaning members 702. The scrubbing material may be, for example, a sponge-like material that promotes or enhances cleaning of the walls of tubes 108 as cleaning members 702 move along the walls. In some embodiments, cleaning members 702 are made from the scrubbing material.

In certain embodiments, base member 704 is a propulsion member for cleaning device 700. For instance, base member 704 may be made of a solid, fluid-resistant (e.g., water-resistant) material such as rubber or hard plastic. The solid, fluid-resistant material in base member 704 may be shaped and sized to provide resistance against fluid flow where the resistance propels cleaning device 700 through tubes 108 (e.g., the fluid resistance of the base member provides propulsion for the cleaning device). In some embodiments, base member 704 includes one or more surfaces that provide resistance against fluid flow. For example, as shown in FIG. 7, base member 704 includes surfaces 710 that are relatively flat and provide resistance against fluid flow with first surface 710A providing resistance against flow in a first direction and second surface 710B providing resistance against flow in a second direction (where the second direction is opposite the first direction). The component base member 704 and its corresponding structural equivalents may be referred to as a “means for propelling a cleaning device through a conduit”.

FIG. 8 depicts a representation of cleaning device 800, according to some embodiments. Cleaning device 800 includes cleaning member 802 attached to base member 804. Cleaning member 802 may include legs 806. Legs 806 may attach cleaning member 802 to base member 804. In certain embodiments, cleaning member 802 is an expandable coil attached to base member 804. For example, cleaning member 802 may be made of flexible or semi-rigid wire or plastic formed into a coil shape with overlapping ends. In such a shape, cleaning member 802 (e.g., the coil) springs or flexes outwards (e.g., expands) from base member 804. As the coil expands, the outward expansion of cleaning member 802 may force contact between edges 808 (such as circular edges) of the cleaning member and the walls of tubes 108. Additionally, cleaning member 802 may have flexibility (such as expansion and contraction flexibility) that allows the cleaning member to freely move through the tubes 108.

In certain embodiments, cleaning member 802 includes openings 810 between legs 806. Openings 810 may allow fluid to flow through cleaning member 802 and reduce drag on cleaning device 800. In some embodiments, openings 810 are sized, shaped, or include features (e.g., protrusions) to promote mixing of the fluid as the fluid flows through the openings. In some embodiments, openings 810 are sized or shaped to maintain an orientation of cleaning device 800 as the device moves through tubes 108. For example, openings 810 may maintain cleaning device 800 in an orientation perpendicular to the walls of tubes 108, as shown in FIG. 8. In some embodiments, cleaning member 802 includes a scrubbing material along edges 808.

Base member 804 may be similar to base member 704, described above. For example, base member 804 may be shaped and sized to provide resistance against fluid flow such that the resistance propels cleaning device 800 through tubes 108. Base member 804 may include one or more surfaces that provide resistance against fluid flow. In some embodiments, cleaning member 802 may include one or more surfaces that provide resistance against fluid flow. For example, as shown in FIG. 8, base member 804 and cleaning member 802 include surfaces that are relatively flat and provide resistance against fluid flow.

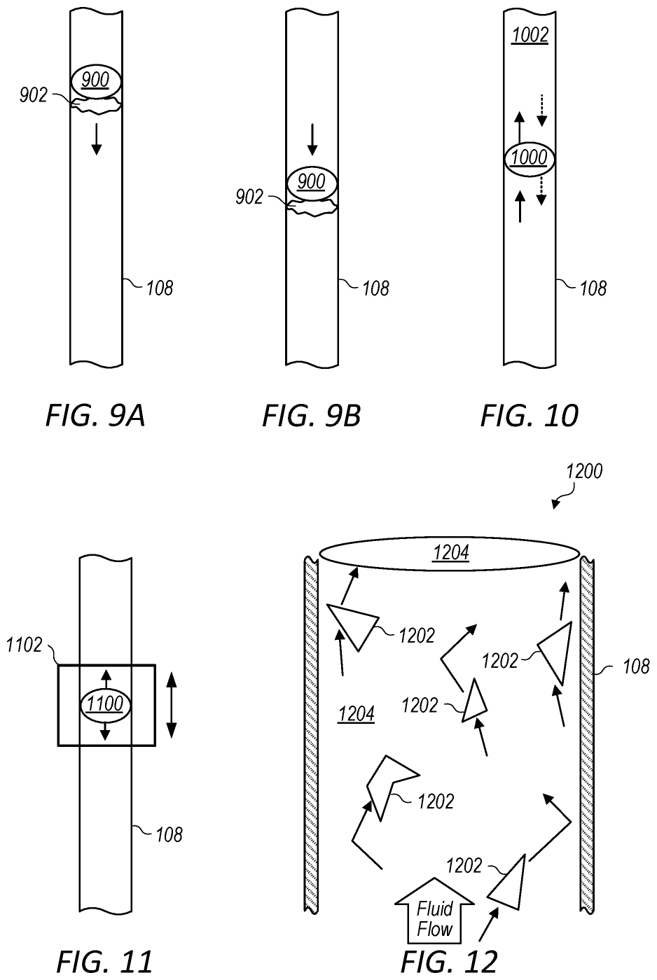

The present inventors have also contemplated methods for moving a cleaning device, such as cleaning device 700 or cleaning device 800, through a bioreactor system with tubes, such as bioreactor 100 with tubes 108. One contemplated embodiment includes utilizing gravity along with flow in tubes 108 to control movement of a cleaning device through the tubes. FIGS. 9A and 9B depict a representation of an example of controlling movement of a cleaning device in tube 108 using gravity and flow, according to some embodiments. Cleaning device 900 may be, for example, a cleaning device such as cleaning device 700 or cleaning device 800, described above. Surface 902 represents a surface of fluid in tube 108. Tube 108 may be a vertical tube or a near-vertical tube (e.g., a substantially vertical tube).

In certain embodiments, cleaning device 900 has a weight that allows the cleaning device to fall with gravity without fluid in tube 108. For example, cleaning device 900 may have enough weight to overcome frictional forces along the wall of tube 108 and the cleaning device 900 will fall without fluid in the tube. Cleaning device 900, however, has a buoyancy that allows the cleaning device to float on surface 902. With a predetermined weight and buoyancy of cleaning device 900, the cleaning device may move up and down with the level of surface 902 in tube 108, as shown in FIGS. 9A and 9B. Thus, controlling the level of surface 902 in tube 108 may be used to control movement of cleaning device 900 in the tube by raising the fluid level to move the cleaning device up in the tube or lowering the fluid level to move the cleaning device down in the tube.

In some embodiments, movement of a cleaning device in a tube is controlled by controlling the direction of flow in the tube. In such embodiments, the cleaning device may not be buoyant such that the cleaning device can reside inside or within the fluid in the tube (e.g., the cleaning device is immersible in fluid). FIG. 10 depicts a representation of an example of controlling movement of a cleaning device in tube 108 with fluid flow, according to some embodiments. Cleaning device 1000 may be, for example, a cleaning device such as cleaning device 700 or cleaning device 800, described above. Cleaning device 1000 is immersed in fluid 1002 in tube 108.

With cleaning device 1000 inside fluid 1002, the cleaning device may be propelled in a desired direction by controlling the direction of flow in the tube, as shown by the arrows in FIG. 10. For example, as shown by the solid line arrows, a flow of fluid in an upwards direction may propel cleaning device 1000 upwards in tube 108 as the fluid flow impacts a propulsion member (such as propulsion member 704 or propulsion member 804, described above) in the cleaning device. Alternatively, as shown by the dashed line arrows, a flow of fluid in a downwards direction may propel cleaning device 1000 downwards in tube 108. It should be noted that controlling the movement of cleaning device 1000 with fluid flow may be implemented in tubes oriented any direction between vertical and horizontal.

The direction of the flow of fluid 1002 in tube 108 may be controlled through various mechanisms. In some embodiments, the flow may be controlled using two pumps, one at either end of the tubes. In some embodiments, a reversible pump is used to control fluid flow and the direction of fluid flow. In some embodiments, a series of valves may be coupled to the tubes to control the direction of fluid flow based on which valves are open. Controlling the direction of fluid flow may be implemented on a single tube or a plurality of tubes (such as all the tubes in a bioreactor). Embodiments are contemplated where control of fluid flow in the tubes is automatic or computer controlled (e.g., through control of pumps and/or valves coupled to the tubes).

FIG. 11 depicts a representation of an example of controlling movement of a cleaning device in tube 108 with a magnetic device, according to some embodiments. Cleaning device 1100 may be, for example, a cleaning device such as cleaning device 700 or cleaning device 800, described above. In certain embodiments, cleaning device 1100 is magnetic. For example, cleaning device 1100 may include magnetic material or be made of magnetic material. Collar 1102 may be placed on the exterior of tube 108. Collar 1102 may be magnetic and magnetically attracted to cleaning device 1100. Thus, collar 1102 may be moved up and down (or any other direction) along tube 108 to control movement of cleaning device 1100 inside tube 108. Collar 1102 may be manually or automatically controlled to control movement of cleaning device 1100 inside tube 108. It should be noted that controlling the movement of cleaning device 1100 with collar 1102 may be implemented in tubes oriented any direction between vertical and horizontal. Additionally, collar 1102 may be any other magnetic device that provides sufficient magnetic attraction to cleaning device 1100 to control movement of the cleaning device along tube 108.

FIG. 12 depicts a representation of cleaning device system 1200, according to some embodiments. Cleaning device system 1200 includes cleaning particles 1202 placed in fluid 1204 inside tube 108. Particles 1202 may be placed in fluid 1204 to provide cleaning of walls inside tube 108. For example, particles 1202 may move along with the flow of fluid 1204 in tube 108 while moving in random/sporadic directions due to mixing, turbulence, or collisions between particles or against the walls of the tube. As particles 1202 interact with the walls of tube 108, the particles may clean the walls in a similar manner to how cholesterol is removed from veins in a human body.

Particles 1202 may be made of flexible material such as rubber, plastic, or a sponge-like material. Particles 1202 may, however, have some rigidity or firmness such that the particles impact the walls of tube 108 with enough force to clean the walls of at least some material. In certain embodiments, particles 1202 have angled edges. For example, particles 1202 may be shaped particles such as polyhedron-shaped particles or dice-shaped particles. In some embodiments, particles 1202 have random shapes (e.g., random polyhedron shapes) to promote random/sporadic movement of the particles after collisions in the tube.

In various embodiments, particles 1202 are small in size and have a flexibility that allows the particles to pass through pumps used in a bioreactor without harming the pumps. For example, in some embodiments, particles have an average diameter of between about 1 cm and about 5 cm. Other diameters may also be contemplated based on pumping requirements, size of valves, or other mechanical requirements.

In some embodiments, catch device 1204 is positioned at a downstream end of a tube. Catch device 1204 may be, for example, a mechanical catch device, a strainer, or a magnetic catch device (if particles 1202 are magnetic). Catch device 1204 may be used to catch particles 1202 before the particles enter a pump or valves at the end of the tube. Particles 1202 may then be reintroduced after the pump or valves or elsewhere in the bioreactor.

The present disclosure includes references to “an “embodiment” or groups of “embodiments” (e.g., “some embodiments” or “various embodiments”). Embodiments are different implementations or instances of the disclosed concepts. References to “an embodiment,” “one embodiment,” “a particular embodiment,” and the like do not necessarily refer to the same embodiment. A large number of possible embodiments are contemplated, including those specifically disclosed, as well as modifications or alternatives that fall within the spirit or scope of the disclosure.

This disclosure may discuss potential advantages that may arise from the disclosed embodiments. Not all implementations of these embodiments will necessarily manifest any or all of the potential advantages. Whether an advantage is realized for a particular implementation depends on many factors, some of which are outside the scope of this disclosure. In fact, there are a number of reasons why an implementation that falls within the scope of the claims might not exhibit some or all of any disclosed advantages. For example, a particular implementation might include other circuitry outside the scope of the disclosure that, in conjunction with one of the disclosed embodiments, negates or diminishes one or more the disclosed advantages. Furthermore, suboptimal design execution of a particular implementation (e.g., implementation techniques or tools) could also negate or diminish disclosed advantages. Even assuming a skilled implementation, realization of advantages may still depend upon other factors such as the environmental circumstances in which the implementation is deployed. For example, inputs supplied to a particular implementation may prevent one or more problems addressed in this disclosure from arising on a particular occasion, with the result that the benefit of its solution may not be realized. Given the existence of possible factors external to this disclosure, it is expressly intended that any potential advantages described herein are not to be construed as claim limitations that must be met to demonstrate infringement. Rather, identification of such potential advantages is intended to illustrate the type(s) of improvement available to designers having the benefit of this disclosure. That such advantages are described permissively (e.g., stating that a particular advantage “may arise”) is not intended to convey doubt about whether such advantages can in fact be realized, but rather to recognize the technical reality that realization of such advantages often depends on additional factors.

Unless stated otherwise, embodiments are non-limiting. That is, the disclosed embodiments are not intended to limit the scope of claims that are drafted based on this disclosure, even where only a single example is described with respect to a particular feature. The disclosed embodiments are intended to be illustrative rather than restrictive, absent any statements in the disclosure to the contrary. The application is thus intended to permit claims covering disclosed embodiments, as well as such alternatives, modifications, and equivalents that would be apparent to a person skilled in the art having the benefit of this disclosure.

For example, features in this application may be combined in any suitable manner. Accordingly, new claims may be formulated during prosecution of this application (or an application claiming priority thereto) to any such combination of features. In particular, with reference to the appended claims, features from dependent claims may be combined with those of other dependent claims where appropriate, including claims that depend from other independent claims. Similarly, features from respective independent claims may be combined where appropriate.

Accordingly, while the appended dependent claims may be drafted such that each depends on a single other claim, additional dependencies are also contemplated. Any combinations of features in the dependent that are consistent with this disclosure are contemplated and may be claimed in this or another application. In short, combinations are not limited to those specifically enumerated in the appended claims.

Where appropriate, it is also contemplated that claims drafted in one format or statutory type (e.g., apparatus) are intended to support corresponding claims of another format or statutory type (e.g., method).

Because this disclosure is a legal document, various terms and phrases may be subject to administrative and judicial interpretation. Public notice is hereby given that the following paragraphs, as well as definitions provided throughout the disclosure, are to be used in determining how to interpret claims that are drafted based on this disclosure.

References to a singular form of an item (i.e., a noun or noun phrase preceded by “a,” “an,” or “the”) are, unless context clearly dictates otherwise, intended to mean “one or more.” Reference to “an item” in a claim thus does not, without accompanying context, preclude additional instances of the item. A “plurality” of items refers to a set of two or more of the items.

The word “may” is used herein in a permissive sense (i.e., having the potential to, being able to) and not in a mandatory sense (i.e., must).

The terms “comprising” and “including,” and forms thereof, are open-ended and mean “including, but not limited to.”

When the term “or” is used in this disclosure with respect to a list of options, it will generally be understood to be used in the inclusive sense unless the context provides otherwise. Thus, a recitation of “x or y” is equivalent to “x or y, or both,” and thus covers 1) x but not y, 2) y but not x, and 3) both x and y. On the other hand, a phrase such as “either x or y, but not both” makes clear that “or” is being used in the exclusive sense.

A recitation of “w, x, y, or z, or any combination thereof” or “at least one of . . . w, x, y, and z” is intended to cover all possibilities involving a single element up to the total number of elements in the set. For example, given the set [w, x, y, z], these phrasings cover any single element of the set (e.g., w but not x, y, or z), any two elements (e.g., w and x, but not y or z), any three elements (e.g., w, x, and y, but not z), and all four elements. The phrase “at least one of . . . w, x, y, and z” thus refers to at least one element of the set [w, x, y, z], thereby covering all possible combinations in this list of elements. This phrase is not to be interpreted to require that there is at least one instance of w, at least one instance of x, at least one instance of y, and at least one instance of z.

Various “labels” may precede nouns or noun phrases in this disclosure. Unless context provides otherwise, different labels used for a feature (e.g., “first conduit,” “second conduit,” “particular conduit,” “given conduit,” etc.) refer to different instances of the feature. Additionally, the labels “first,” “second,” and “third” when applied to a feature do not imply any type of ordering (e.g., spatial, temporal, logical, etc.), unless stated otherwise.

The phrase “based on” or is used to describe one or more factors that affect a determination. This term does not foreclose the possibility that additional factors may affect the determination. That is, a determination may be solely based on specified factors or based on the specified factors as well as other, unspecified factors. Consider the phrase “determine A based on B.” This phrase specifies that B is a factor that is used to determine A or that affects the determination of A. This phrase does not foreclose that the determination of A may also be based on some other factor, such as C. This phrase is also intended to cover an embodiment in which A is determined based solely on B. As used herein, the phrase “based on” is synonymous with the phrase “based at least in part on.”

Within this disclosure, different entities (which may variously be referred to as “units,” “circuits,” other components, etc.) may be described or claimed as “configured” to perform one or more tasks or operations. This formulation—[entity] configured to [perform one or more tasks]—is used herein to refer to structure (i.e., something physical). More specifically, this formulation is used to indicate that this structure is arranged to perform the one or more tasks during operation. A structure can be said to be “configured to” perform some task even if the structure is not currently being operated. Thus, an entity described or recited as being “configured to” perform some task refers to something physical, such as a device, circuit, a system having a processor unit and a memory storing program instructions executable to implement the task, etc. This phrase is not used herein to refer to something intangible.

In some cases, various units/circuits/components may be described herein as performing a set of task or operations. It is understood that those entities are “configured to” perform those tasks/operations, even if not specifically noted.

The term “configured to” is not intended to mean “configurable to.” An unprogrammed FPGA, for example, would not be considered to be “configured to” perform a particular function. This unprogrammed FPGA may be “configurable to” perform that function, however. After appropriate programming, the FPGA may then be said to be “configured to” perform the particular function.

For purposes of United States patent applications based on this disclosure, reciting in a claim that a structure is “configured to” perform one or more tasks is expressly intended not to invoke 35 U. S.C. § 112(f) for that claim element. Should Applicant wish to invoke Section 112(f) during prosecution of a United States patent application based on this disclosure, it will recite claim elements using the “means for” [performing a function] construct.

What is claimed is:

1. A device configured to clean a conduit associated with growing biological organisms, comprising:
at least one base member, wherein the at least one base member includes at least one surface that provides resistance against fluid flow in order to propel the device in a direction of the fluid flow; and
at least one cleaning member made of semi-rigid material coupled to the at least one base member, the at least one cleaning member having at least one circular edge, wherein the at least one cleaning member extends outwards from the at least one base member such that the at least one circular edge makes contact with an inner wall of the conduit, and wherein the at least one cleaning member includes at least one opening to allow fluid flow through the at least one cleaning member.

2. The device of claim 1, wherein the at least one circular edge of the at least one cleaning member makes contact with the inner wall of the conduit along a circumference of the at least one circular edge.

3. The device of claim 1, wherein the at least one cleaning member and the at least one base member are shaped such that the device maintains a proper orientation in the conduit.

4. The device of claim 1, further comprising a scrubbing material positioned along the at least one circular edge of the at least one cleaning member.

5. The device of claim 1, further comprising at least two cleaning members coupled to the at least one base member.

6. The device of claim 1, wherein the at least one cleaning member is a conical-shaped member, and wherein the at least one base member is a solid object coupled to the conical-shaped member.

7. The device of claim 1, wherein the at least one cleaning member is an expandable coil, and wherein the at least one base member is coupled to the expandable coil by one or more legs.

8. The device of claim 1, wherein the at least one base member has a buoyancy that allows the device to float on a surface of the fluid in the conduit.

9. The device of claim 1, wherein the at least one surface of the at least one base member is configured to provide resistance against the fluid flow in a first direction and a second direction, the second direction being opposite the first direction.

10. The device of claim 1, further comprising a magnet coupled to the at least one base member, wherein the magnet is configured to be attracted to an external magnetic device positioned outside the conduit such that movement of the external magnetic device moves the device along the conduit.

11. A method for cleaning a conduit associated with growing biological organisms, comprising:
placing a device comprising at least one cleaning member coupled to at least one base member in the conduit, the at least one cleaning member being made of semi-rigid material, wherein the at least one cleaning member has at least one circular edge and the at least one cleaning member extends outwards from the at least one base member such that the at least one circular edge makes contact with an inner wall of the conduit, wherein the at least one cleaning member includes at least one opening through the at least one cleaning member, and wherein the at least one base member includes at least one surface that provides fluid flow resistance; and
moving the device along the conduit to remove biological organisms from the inner wall of the conduit using the at least one cleaning member.

12. The method of claim 11, wherein moving the device along the conduit includes providing a force against the at least one surface on the at least one base member using a flow of fluid in the conduit.

13. The method of claim 12, further comprising controlling moving the device by controlling the flow of fluid in the conduit.

14. The method of claim 11, wherein the at least one surface of the at least one base member has a buoyancy that allows the device to float on a fluid surface, and wherein moving the device along the conduit includes raising or lowering a fluid level in the conduit.

15. The method of claim 11, wherein the at least one base member is immersed in the fluid, and wherein moving the device along the conduit includes raising or lowering a fluid level in the conduit.

16. The method of claim 11, wherein the device includes a magnet coupled to the at least one base member and an external magnetic device that is attracted to the magnet, wherein moving the external magnetic device along the conduit moves the at least one base member along the conduit.

17. The method of claim 16, wherein the external magnetic device is a magnetic collar that moves along an outer wall of the conduit.

18. The method of claim 12, further comprising promoting mixing in the fluid flowing through the at least one opening in the at least one cleaning member based on a size of the at least one opening, a shape of the at least one opening, or features placed in the at least one opening.

19. A device configured to clean a conduit associated with growing biological organisms, comprising:
a base member made of solid, fluid-resistant material, wherein at least one surface of the base member provides resistance against fluid flow in the conduit; and
a cleaning member made of semi-rigid material, the cleaning member having a conical shape, wherein the cleaning member is coupled to the base member at an apex of the conical shape, wherein the cleaning member has a circular opening formed by the semi-rigid material at an end of the conical shape distal from the apex, wherein at least one edge of the circular opening of the cleaning member makes contact with an inner wall of the conduit when the cleaning member is positioned in the conduit, and wherein the cleaning member includes at least one opening through the semi-rigid material.

20. The device of claim 19, further comprising a second cleaning member made of semi-rigid material, the second cleaning member having a conical shape, wherein the second cleaning member is coupled to a side of the base member opposite to the cleaning member.
